(19) United States
(12) Patent Application Publication
(10) Pub. No.: US 2006/0064114 A1
(43) Pub. Date: Mar. 23, 2006

(54) Wire to remove intravascular foreign body and medical instrument

(75) Inventors: Hideshi Obitsu, Shizuoka (JP); Takeshi Kanamaru, Kanagawa (JP)

Correspondence Address: BUCHANAN INGERSOLL PC;(INCLUDING BURNS, DOANE, SWECKER & MATHIS), POST OFFICE BOX 1404, ALEXANDRIA, VA, 22313-1404, US

(73) Assignee: Terumo Kabushiki Kaisha, Tokyo (JP)

(21) Appl. No.: 11/232,021

(22) Filed: Sep. 22, 2005

(30) Foreign Application Priority Data
Sep. 22, 2004 (JP) 2004-275991

(51) Int. Cl. A61B 17/26

(52) U.S. Cl. 606/113

(57) ABSTRACT

A wire to remove an intravascular foreign body includes a flexible long wire body, a foreign body capturing portion to capture a foreign body in a vessel, and an operating wire to deform the foreign body capturing portion. The foreign body capturing portion includes a first capturing part and a second capturing part. The first capturing part is formed from a filament which assumes a helical shape in its natural state and is arranged on the forward end of the wire body. The second capturing part is formed from a filament which assumes a helical shape in its natural state and is arranged on the forward end of the first capturing part. The first and second capturing parts together form a rearwardly open space for capturing the foreign body when the wire is moved rearwardly relative to the foreign body. The second capturing part is deformable such that the distance between its adjacent filaments decreases upon operation of the operating wire. The wire is capable of surely capturing and removing a foreign body in the vessel.

### BACKGROUND OF THE INVENTION

The present invention relates to a wire and method to remove an intravascular foreign body and a medical instrument.

The vital statistics of population published by the Ministry of Health, Labor, and Welfare indicates that cancer dominates in the cause of Japanese death and heart disease and cerebral apoplexy come second and third. The increasing deaths and sequelae due to cerebral apoplexy urgently demand to establish its therapeutic method.

A recent development in therapy of cerebral apoplexy is thrombolysis that employs a thrombolytic agent to cure brain infarction in its acute phase. It is effective but its effectiveness is limited. That is, the thrombolytic agent takes a long time for thrombolysis or produces smaller thrombi that scatter to form new emboli. In addition, it has been found by doctors that some emboli are insoluble by treatment with a thrombolytic agent.

It has been proved in the U.S. and Europe that the probability to save lives and reduce sequelae would be high if the blood flow is resumed within 3 hours after the onset of cerebral apoplexy. Thus, there is a strong demand for development of a new medical instrument that can be inserted into a cerebral vessel to remove the thrombus directly.

An example of such a medical instrument is one which has a basket (to capture a foreign body) which is movable (from its retreated position to its expanded position) relative to the sheath (or catheter). Refer to PCT Application No. 2002-516139 (U.S. Pat. No. 6,096,053).

### SUMMARY OF THE INVENTION

It is desirable to provide a wire to surely capture and remove an intravascular foreign body.

The present invention provides in a wire to remove an intravascular foreign body including a flexible long wire body having a forward end, a foreign body capturing portion for capturing a foreign body in a vessel, and an operating wire to deform the foreign body capturing portion. The foreign body capturing portion has a first capturing part and a second capturing part. The first capturing part is formed from a filament which assumes a helical shape in its natural state and is arranged on the forward end of the wire body. The second capturing part is formed from a filament which assumes a helical shape in its natural state and is arranged on the forward end of the first capturing part. The second capturing part is deformable such that the distance between its adjacent filament turns decreases upon operation of the operating wire.

The present invention also provides a wire to remove an intravascular foreign body including a flexible long wire body having a forward end, a foreign body capturing portion for capturing a foreign body in a vessel, and an operating wire to deform the foreign body capturing portion. The foreign body capturing portion includes a first capturing part and a second capturing part. The first capturing part is placed at the forward end of the wire body, and in its natural state, includes a first group of loop wires arranged at certain intervals in a lengthwise direction of the wire body. The second capturing part is placed at the forward end of the first capturing part, and in its natural state, includes a second group of loop wires arranged at certain intervals in the lengthwise direction of the wire body. The second capturing part is deformable such that the distance between the adjacent loop wires of the second group decreases upon operation of said operating wire.

Additionally, the present invention provides a medical instrument including the wire to remove an intravascular foreign body as defined above and a catheter having a lumen for receiving therein the wire to remove an intravascular foreign body.

The present invention further provides a wire to remove an intravascular foreign body which comprises a flexible long wire body having a forward end thereof, a foreign body capturing portion extending from the forward end for capturing a foreign body, and an operating wire to deform the foreign body capturing portion. The foreign body capturing portion includes a plurality of loops spaced apart in a lengthwise direction of the wire body and defining a rearwardly open space for receiving a foreign body as the foreign body capturing portion is moved rearwardly relative to the foreign body. The operating wire is operable to draw the loops toward one another to resist escape of the captured foreign body from the space.

The invention also provides a method of removing an intravascular foreign body from a patient's vessel, comprising the steps of:

A) introducing a catheter into the vessel;

B) introducing through the catheter a wire body having at its forward end a foreign body capturing portion which comprises a plurality of loops spaced apart in a lengthwise direction of the wire body and defining a rearwardly open space;

C) positioning the foreign body capturing portion forwardly of the foreign body;

D) moving the foreign body capturing portion rearwardly to cause the foreign body to enter and be captured in the space; and

E) manipulating an operating wire operably connected to the loops to draw at least some adjacent loops toward one another to resist escape of the foreign body from the space.

According to the present invention, the capturing and removing an intravascular foreign body is surely achieved by deforming the foreign body capturing part such that the distance between its adjacent filaments (forming loops) decreases.

### BRIEF DESCRIPTION OF THE DRAWINGS

FIG. 1 is a partial longitudinal sectional view showing the wire to remove an intravascular foreign body (in its natural state) pertaining to the first embodiment of the present invention;

FIG. 2 is a partial longitudinal sectional view showing the wire to remove an intravascular foreign body (in its contracted state) shown in FIG. 1;

FIG. 3 is a diagram illustrating how to use the wire to remove an intravascular foreign body shown in FIG. 1;

FIG. 4 is a diagram illustrating how to use the wire to remove an intravascular foreign body shown in FIG. 1;

FIG. 5 is a diagram illustrating how to use the wire to remove an intravascular foreign body shown in FIG. 1;

FIG. 6 is a diagram illustrating how to use the wire to remove an intravascular foreign body shown in FIG. 1;

FIG. 7 is a diagram illustrating how to use the wire to remove an intravascular foreign body shown in FIG. 1;

FIG. 8 is a diagram illustrating how to use the wire to remove an intravascular foreign body shown in FIG. 1;

FIG. 9 is a partial longitudinal sectional view showing the wire to remove an intravascular foreign body (in its natural state) pertaining to the second embodiment of the present invention; and

FIG. 10 is a partial longitudinal sectional view showing the wire to remove an intravascular foreign body (in its contracted state) shown in FIG. 9.

### DETAILED DESCRIPTION OF THE PREFERRED EMBODIMENTS

A detailed description is given below of the wire to remove an intravascular foreign body and the medical instrument of the present invention with reference to the preferred embodiments shown in the accompanying drawings.

### First Embodiment

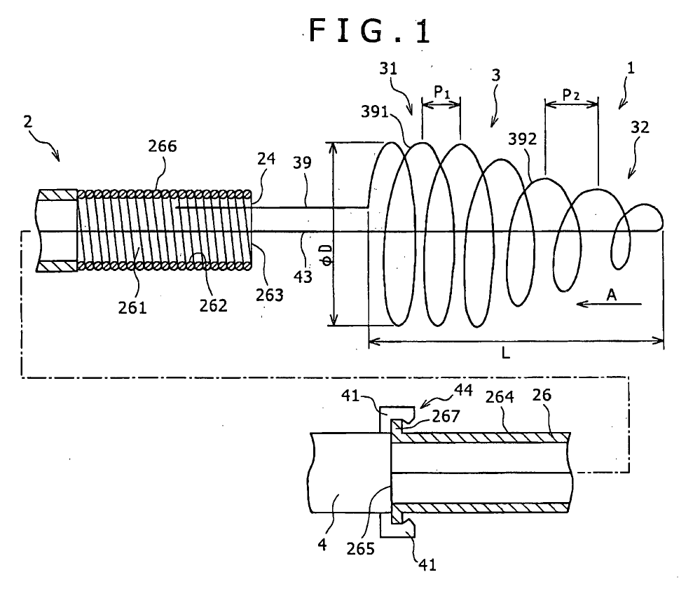

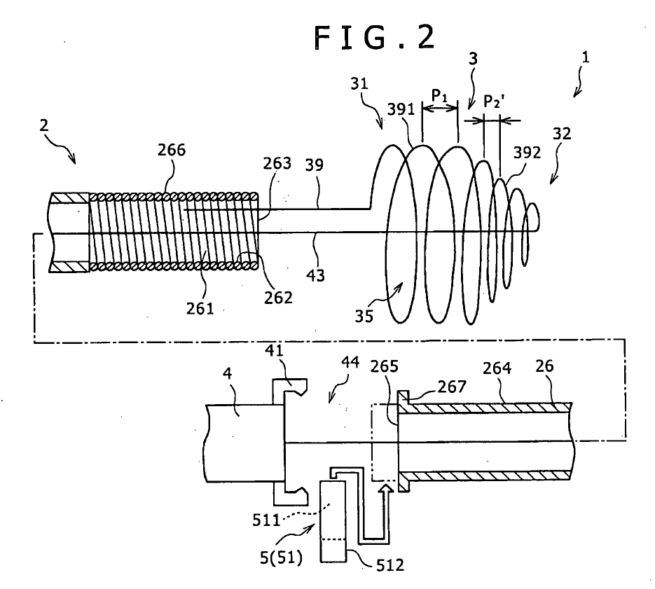

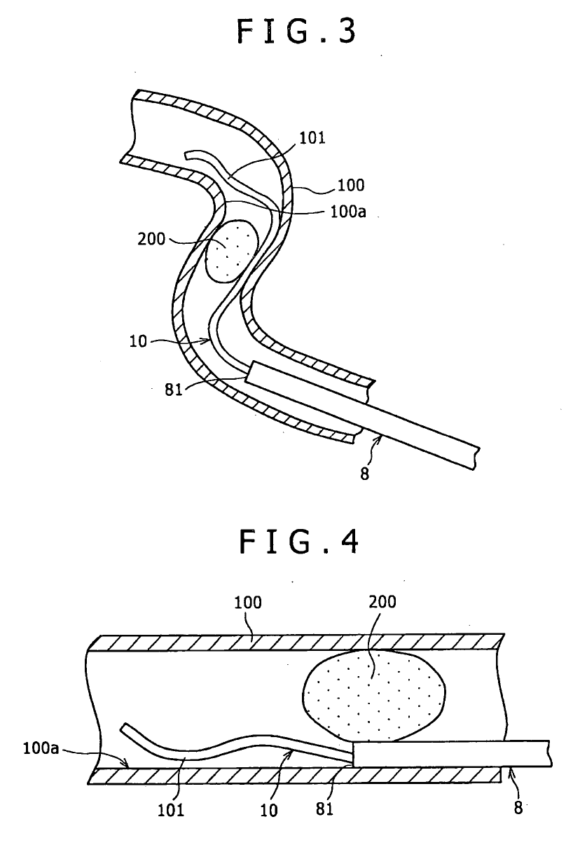

FIG. 1 is a partial longitudinal sectional view showing the wire to remove an intravascular foreign body (in its natural state) pertaining to the first embodiment of the present invention. FIG. 2 is a partial longitudinal sectional view showing the wire to remove an intravascular foreign body (in its contracted state) shown in FIG. 1. FIGS. 3 to 8 are diagrams each illustrating how to use the wire to remove an intravascular foreign body shown in FIG. 1.

Incidentally, the terms “base end (proximal end)” and “forward end (distal end)” in the following description are defined as follows. In FIGS. 1 and 2, the left side is “base end (proximal end)” and the right side is “forward end (distal end)”, and in FIGS. 3 to 8, the right side is “base end (proximal end)” and the left side is “forward end (distal end)”.

FIG. 1 shows the wire to remove an intravascular foreign body (named 1), which is intended to capture and remove a foreign body (such as thrombus and clot) which causes embolism in the vessel. A foreign body will be referred to as “embolus 200” hereinafter.

The wire 1 to remove an intravascular foreign body includes a long wire body 2, a foreign body capturing part or portion 3 attached to the forward end of the wire body 2, and an operating wire 43 to deform the foreign body capturing part 3. Each part is constructed as described in the following.

The wire body 2 shown in FIG. 1 includes a tube 26 and a coil 266 arranged on (attached or fixed to) the forward end of the tube 26. The wire body 2 has adequate rigidity and resilience (flexibility) over its entire length.

Thus, the forward end of the tube 26 (including the coil 266) can have rigidity and flexibility by providing the coil 266.

The parts constructing the wire body 2 may be formed from any materials without specific restrictions, such as metallic and plastic materials, which may be used alone or in combination.

The wire body 2 may vary in length (the total length of the tube 26 and the coil 266) depending on the position and size of the blood vessel to which it is applied. A preferred length ranges from 500 to 4000 mm usually, and more preferred length ranges from 1500 to 2200 mm.

The wire body 2 (or the tube 26) may also vary in thickness (outside diameter) depending on the position and size of the vessel 100 to which it is applied. A preferred outside diameter is usually 0.1 to 2.0 mm on average, and more preferred length ranges from 0.25 to 0.9 mm.

The wire body 2 should preferably be composed of a first part (which is comparatively hard and is placed at the base end), a third part (which is comparatively soft and is placed at the forward end), and a second part (which is variable in flexibility and is placed at the intermediate position between the first part and the third part). In other words, the wire body 2 should preferably be formed in such a way that it gradually decreases in rigidity (flexural and torsional rigidity) in going from its base end to its forward end. The gradually changing rigidity permits the manual manipulation to be certainly transmitted to the forward end 24 of the wire body 2. With such properties (that make the forward end 24 flexible), the wire body 2 easily proceeds and bends in the vessel 100 without damaging the vessel 100. Such bodies permit the wire body 2 to transmit its twisting motion and its pushing motion while preventing kinking (or flexing). This contributes to higher safety.

The wire body 2 may have a coating layer on its outer surface for reduction of friction with the inside of the catheter 8 (mentioned later). The coating layer permits smooth insertion into and removal from the catheter. The coating layer may be formed from a fluorocarbon resin (such as polytetrafluoroethylene or Teflon® or a hydrophilic polymer which exhibits lubricity in its wet state.

As shown in FIGS. 1 and 2, the foreign body capturing part 3 is attached (fixed) to the forward end of the internal periphery 262 defining the bore 261 of the coil 266. The foreign body capturing part 3 projects from the forward opening 263 of the coil 266 in the lengthwise direction of the tube 26 and includes first and second capturing parts 31 and 32.

No specific restriction is imposed on the method of fixing the foreign body capturing part 3 to the coil 266 (the wire body 2). One method may include winding the base end of a filament 39 of the first capturing part 31 around the forward end of the wire body 2 and then performing brazing, welding, or adhesion with an adhesive. See FIG. 1.

In this embodiment, at the forward end of the wire body 2 the coil 266 covers the fixing part (brazed part) where the foreign body capturing part 3 is fixed to the wire body 2. The outer surface of the coil 266 is smooth, which ensures higher safety. The coil 266 should preferably be formed by a helically wound platinum wire.

The first capturing part 31 is attached to the forward end of the wire body 2, and the second capturing part 32 is arranged on the forward end of the first capturing part 31.

The first capturing part 31 is made of a filament 391 which assumes a helical coil shape in its natural state. The second capturing part 32 is made of a filament 392 which assumes a helical coil shape in its natural state, with the coil decreasing in loop diameter in going forward. The second capturing part 32 is more flexible than the first capturing part 31.

The wire 1 to remove an intravascular foreign body, which is constructed as mentioned above, permits the second capturing part 32 to deform in such a way that the distance between the adjacent loops or turns of the filament 392 decreases, as shown in FIG. 2, when the operating wire 43 is operated.

According to this embodiment, the first capturing part 31 has a larger loop diameter than the second capturing part 32. Therefore, the first capturing part 31 surely captures (holds) therein the embolus 200 during operation to capture a foreign body.

The foreign body capturing part 3 includes integrally formed filaments 39, 391, and 392 and the operating wire 43 which are formed from a single wire. This structure reduces the number of constituent parts. The wire 1 to remove an intravascular foreign body is flexible, so that it easily reaches any small part in the vessel.

The foreign body capturing part 3 could alternatively be formed from two parts. One part is a single wire constituting the filaments 39, 391, and 392, and the other part is a single wire constituting the operating wire 43. These wires may be bonded together (or fixed by twisting) at the forward end of the foreign body capturing part 3.

In this embodiment, the filament 391 and the filament 392 are the same filament.

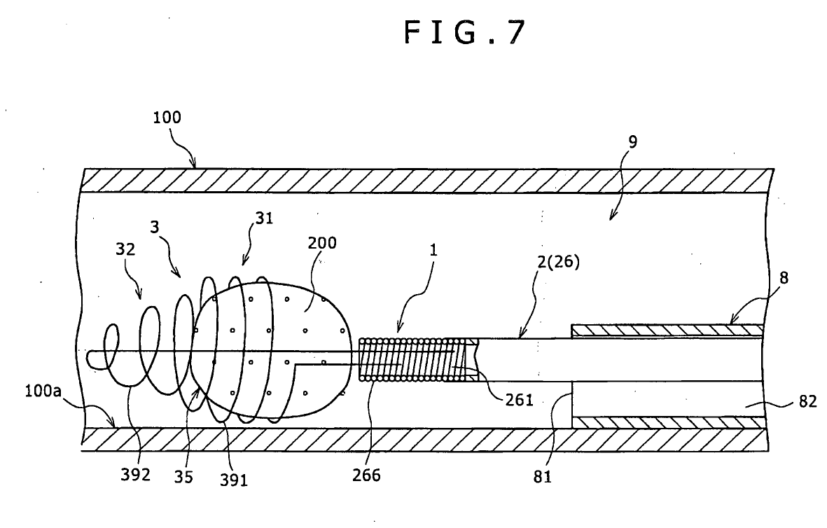

According to this embodiment, the first capturing part 31 and the second capturing part 32 are adjacent to each other, and the space formed by the first capturing part 31 and the space formed by the second capturing part 32 communicate with each other, so that they form a foreign body capturing space 35 which is rearwardly open, i.e., open toward the wire body 2, as shown in FIG. 7.

When the operating member 4 (located at the base end of the operating wire 43) is operated by being retracted (i.e., moved to the left in FIG. 2), the capturing part 32 is caused to deform in such a way that the adjacent filament turns (loops) thereof draw near to each other as shown in FIG. 2. Deformation in this manner prevents the embolus 200 (which might be a soft one) from slipping off through the gap between the adjacent turns of the filament 392. This ensures the capturing of the embolus 200.

The term “contracted state” will be used hereinafter to denote the state in which the second capturing part 32 is deformed such that the adjacent turns of the filament 392 thereof draw near to each other.

In the contracted state, the first capturing part 31 may also be deformed in such a way that the adjacent turns of the filament 391 get near to each other. However, it is so designed as to deform less than the second capturing part 32.

The foreign body capturing part 3 is not specifically restricted in the distance between the adjacent filament turns thereof. It may assume any shape so long as it can capture the embolus 200. It is desirable in the natural state that the average distance (indicated by P2 in FIG. 1) between the adjacent filament turns of the second capturing part 32 should be larger than the average distance (indicated by P1 in FIG. 1) between the adjacent filament turns of the first capturing part 31.

Thus, the foreign body capturing part 3 surely changes from its natural state into its contracted state, and the foreign body capturing part 3 in its contracted state surely captures (removes) the embolus 200.

If the embolus 200 in the cerebral blood vessel is to be captured, the average distance P2 between the adjacent turns of the filament 392 of the second capturing part 32 in its natural state should preferably be about 1 to 20 mm, more preferably about 1 to 8 mm, although it is not specifically restricted.

The average distance P1 in the lengthwise direction of the wire body 2 between the adjacent turns of the filament 391 of the first capturing part 31 in its natural state should preferably be about 0.5 to 15 mm, more preferably about 1 to 5 mm, although the distance is not specifically restricted.

In order to capture the embolus 200, the operating wire 43 (the operating member 4) is operated so that the foreign body capturing part 3 is contracted. At this time, the second capturing part 32 is deformed to such an extent that the average distance in the lengthwise direction of the wire body 2 (indicated by P2′ in FIG. 2) between the adjacent turns of the filament 392 of the second capturing part 32 becomes smaller than the average distance P1 between the adjacent turns of the filament 391. In this way the foreign body capturing part 3 (or the foreign body capturing space 35) can surely capture the embolus 200 which might be comparatively soft.

If the embolus 200 in the cerebral blood vessel is to be captured, the average distance P2′ between the adjacent filament turns of the second capturing part 32 in its contracted state should preferably be about 0.1 to 10 mm, more preferably about 0.1 to 3 mm, although the distance is not specifically restricted.

The foreign body capturing part 3 is not specifically restricted in its maximum loop diameter (indicated by φD in FIG. 1) so long as it is large enough to capture the embolus 200. If the embolus 200 in the cerebral blood vessel is to be captured, the maximum loop diameter should preferably be about 1 to 5 mm, more preferably about 2 to 4 mm.

The foreign body capturing part 3 (the filaments 39, 391, and 392) is not specifically restricted in the filament diameter (thickness) so long as it is large enough to capture the embolus 200. If the embolus 200 in the cerebral blood vessel is to be captured, the filament diameter should preferably be about 0.04 to 0.30 mm, more preferably about 0.04 to 0.12 mm.

The foreign body capturing part 3 is not specifically restricted in its length (indicated by L in FIG. 1) so long as it is long enough to capture the embolus 200. The desired length varies depending on the position and size of the vessel to which it is applied. If the embolus 200 in the cerebral blood vessel is to be captured, the length of the capturing part 3 in its natural state should preferably be about 1 to 30 mm, more preferably about 5 to 15 mm.

The foreign body capturing part 3 is not specifically restricted in the number of turns (i.e., loops) so long as it has as many turns as necessary to capture the embolus 200. If the embolus 200 in the cerebral blood vessel is to be captured, the number of turns should preferably be about 3 to 16, more preferably about 6 to 10.

The capturing part 3 should preferably be formed from any radiopaque material which is not specifically restricted. It includes, for example, gold, platinum, platinum-iridium alloy, tungsten, tantalum, palladium, lead, silver, and their alloys and compounds.

Such radiopaque materials facilitate manipulation (to capture the embolus 200 by the capturing part 3) with the help of X-ray radioscopy.

The capturing part 3 should preferably be formed from an alloy which exhibits pseudo-elasticity at the living body temperature (about 37° C.). (Such an alloy includes one which exhibits superelasticity, which will be referred to as “superelastic alloy” hereinafter.)

The alloy which exhibits pseudo-elasticity (which will be referred to as “pseudo-elastic alloy” hereinafter) includes those which show any stress-strain curve under tension and which is able to or difficult to measure clear transformation points. Examples of those that can measure clear transformation are As, Af, Ms, and Mf. It includes those which undergo large deformation (strain) under stress and restore their original shape after stress has been removed.

The superelastic alloy is included in the pseudo-elastic alloy. Preferred examples of the superelastic alloy include Ni—Ti alloy containing 49 to 52 atom % of Ni, Cu—Zn alloy containing 38.5 to 41.5 wt % of Zn, Cu—Zn—X alloy containing 1 to 10 wt % of X (where X denotes at least one species of Be, Si, Sn, Al, and Ga), and Ni—Al alloy containing 36 to 38 atom % of Al. Of these alloys, the Ni—Ti alloy is particularly preferable.

The foreign body capturing part 3 formed from the above-mentioned pseudo-elastic alloy has good flexibility and an ability to recover from bending. The good recoverability prevents the foreign body capturing part 3 from remaining in a bent shape.

The foreign body capturing part 3 may have a coating layer on its outer surface for reduction of friction with the inside of the catheter 8. The coating layer permits smooth insertion into and removal from the catheter. The coating layer may be formed from a fluorocarbon resin (such as polytetrafluoroethylene or Teflon® or a hydrophilic polymer which becomes lubricious in a wet condition.

The operating member 4 is designed to move the second capturing part 32 in the lengthwise direction of the wire body 2.

The operating member 4 is connected to the second capturing part 32 through the operating wire 43 which passes through the wire body 2.

Thus, the operating member 4 (the operating wire 43) facilitates the operation of the second capturing part 32.

The wire 1 to remove an intravascular foreign body, which is constructed as mentioned above, causes the foreign body capturing part 3 to change from its natural state into its contracted state through the following steps.

In the first step, the operating member 4 is retracted, i.e., is moved toward the base end (the direction of arrow A in FIG. 1). This operation causes the foreign body capturing part 3 to act such that the forward end of the second capturing part 32 moves toward the first capturing part 31 (in the direction of arrow A in FIG. 1). At this time, the second capturing part 32 is deformed such that the average distance P2 between filament turns decreases to the average distance P2′. In other words, the foreign body capturing part 3 changes from its natural state (FIG. 1) into its contracted state (FIG. 2).

The wire 1 to remove an intravascular foreign body, which is constructed as mentioned above, works in the following manner. The foreign body capturing part 3 in its contracted state forms the foreign body capturing space 35 in which the embolus 200 is captured in the vessel 100.

The second capturing part 32 changes into its contracted state when it captures the foreign body (the embolus 200), so that it hangs on to the embolus 200 and prevents the embolus 200 from slipping off through the gap between the filaments 391 and 392. In this way the capturing part surely captures and removes the embolus 200.

The foreign body capturing part 3 may be provided with a means for preventing the embolus 200 (which has been captured) from slipping off from it. Such an anti-slipping means permits the capturing part 3 to surely hold (capture) the embolus 200.

The anti-slipping means is not specifically restricted. It may be formed by coating with an elastic material (such as rubber) having a comparatively high coefficient of friction or by sand blasting which produces fine rough surfaces or surface irregularities.

Other anti-slipping means include fine fibers attached to the foreign body capturing part 3 and the wire body 2. Such fine fibers make it easier to capture the embolus 200 owing to increased friction between the foreign body capturing part 3 and the embolus 200. The increased friction prevents the captured embolus 200 from slipping off from the foreign body capturing part 3, thereby ensuring the capturing of the embolus 200.

The foreign body capturing part 3 may have a coating which swells upon contact with a liquid. Such a coating reduces the average filament distance P1 and the average filament distance P2′ in the contracted state. This helps surely hold the captured embolus 200.

The swellable coating may be common gel (such as PVA in general use). It also includes hydrogel foam substance, hydrophilic macroporous substance, polymeric hydrogel foam substance, and water-swellable foam matrix. The last one is a macroporous solid as a polymer or copolymer formed from a polymerizable hydrophilic olefin monomer containing a foam stabilizer and free radicals crosslinked with a multiolefin crosslinking agent (up to 10 wt %).

As shown in FIG. 1, the wire 1 to remove an intravascular foreign body has a means that prevents the foreign body capturing part 3 in its natural state from changing into its contracted state. In other words, it has means 44 to keep the foreign body capturing part 3 in its natural state.

The natural state retaining means 44 includes a projection 267 and a claw 41 that engages with the projection 267. The projection 267 is formed from the peripheral part 264 of the tube 26 (which is bent in the radial direction) in the vicinity of the base opening 265 of the tube 26. The claw 41 is formed in the vicinity of the forward end of the operating member 4. In other words, the natural state retaining means 44 includes the projection 267 and the claw 41.

The claw 41 releases the projection 267 by elastic deformation of the claw.

The natural state retaining means 44 constructed as mentioned above permits the operating member 4 to move toward the base end (i.e., to the left in FIG. 1) when the foreign body capturing part 3 is in its natural state, thereby preventing the foreign body capturing part 3 from assuming its contracted state inadvertently.

As shown in FIG. 2, the wire 1 to remove an intravascular foreign body has a contracted state retaining means 5 which retains the foreign body capturing part 3 in its contracted state.

The contracted state retaining means 5 comprises a clip 51, e.g., which has a notch 511 formed in its radial direction.

While the capturing part is in its contracted state, the clip 51 is mounted on the operating wire 43 by means of the notch 511 (in the direction of arrow in FIG. 2), with the side 512 of the clip 51 in contact with the base end of the tube 26, as shown in FIG. 2. In this state, the width of the notch is so narrow that the operating wire 43 is secured to the clip 51 and is not able to move relative to the clip 51.

The contracted state retaining means 5 (or the clip 51) constructed as mentioned above prevents the operating wire 43 from moving in the forward direction and thus prevents the foreign body capturing part 3 in its contracted state from opening (or assuming its natural state) inadvertently.

The clip 51 may be formed from any metallic material and plastic material alone or in combination.

The wire 1 to remove an intravascular foreign body, which is constructed as mentioned above, may be used as a medical tool to remove a thrombus and an intravascular foreign body. It is very useful as a medical instrument to cure cerebral apoplexy. It is also useful as a medical instrument for ischemic heart diseases (venous and arterial embolus) which are not easily cured by a thrombolytic agent. It is used to remove various foreign bodies from the vessel.

The wire 1 to remove an intravascular foreign body, which has been mentioned above, is constructed such that the foreign body capturing part 3 has only one continuous helical filament structure 391, 392. However, the present invention is not limited to it. The foreign body capturing part 3 may have two (or more) continuous helical filament structures. The modified construction makes the filaments move complicated and tight to hang on to the embolus 200 more effectively and prevent the captured embolus 200 from slipping off through the gap between adjacent filament turns. This ensures the capturing and removal of the embolus 200.

In that case, the two helical filament structures may be wound in the same direction or in mutually opposite directions (the latter being preferable). The modified construction will hang on to the embolus 200 more effectively. This ensures the capturing and removal of the embolus 200.

The medical instrument 9 according to the present invention includes the wire 1 to remove an intravascular foreign body and the catheter 8 which has the lumen 82 formed therein.

A detailed description is given below of one way of using the medical instrument 9 provided with the wire 1 to remove an intravascular foreign body.

FIG. 3 depicts the vessel 100 which is clogged with the embolus 200 (such as thrombus) which hinders blood flow. The embolus 200 is almost immobile because it is pushed against the inner wall 100a of the vessel 100 by blood pressure.

The first step is to insert the catheter (microcatheter) 8 and a guide wire 10 (which has been passed through the lumen 82 of the catheter 8) into the vessel 100. The second step is to project the guide wire 10 from the forward open end 81 of the catheter 8 beyond the embolus 200. In other words, the second step is carried out such that the forward end 101 of the guide wire 10 passes through the gap between the embolus 200 and the inner wall 100a of the vessel 100 and gets over the embolus 200. This operation can be easily accomplished by using the guide wire 10 (microguide wire) which has good lubricity.

After the forward end 101 of the guide wire 10 has gotten over the embolus 200, the catheter 8 is advanced relative to the guide wire 10, so that the forward end of the catheter 8 gets in the gap between the embolus 200 and the inner wall 100a of the vessel 100, as shown in FIG. 4. This operation is easy because the forward end of the catheter 8 smoothly gets in the gap along the guide wire 10.

According to the conventional therapy, the above-mentioned stage is followed by injection of a thrombolytic agent through the catheter 8. However, it is often experienced by doctors that there are emboli 200 which are not dissolved by a thrombolytic agent or dissolution by a thrombolytic agent takes a long time. The present invention is effective in such a case.

The step shown in FIG. 4 is followed by the next step in which the guide wire 10 is removed and the wire 1 to remove an intravascular foreign body is inserted into the lumen 82 of the catheter 8.

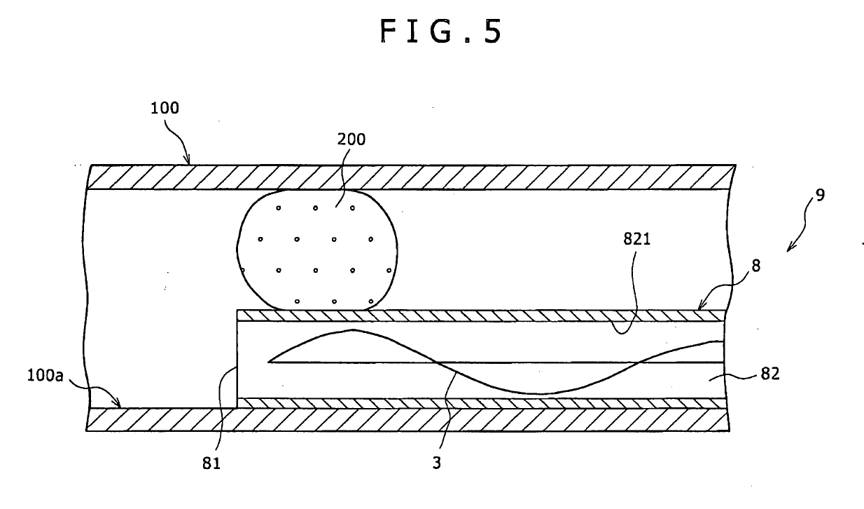

As shown in FIG. 5, the foreign body capturing part 3 is in an elongated straight state because it is confined by the inner wall 821 of the lumen 82 and it is stretched as the operating wire 43 is moved toward the forward end. Being able to assume the elongated state, the foreign body capturing part 3 easily passes through the lumen 82. For the foreign body capturing part 3 to assume the elongated state, it is necessary that the operating wire 43 be relaxed between the operating member 4 and the foreign body capturing part 3.

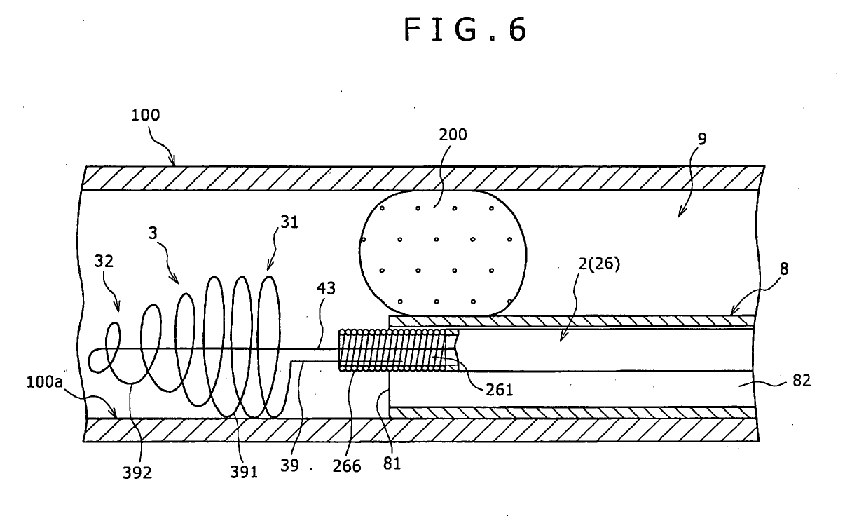

Then, the foreign body capturing part 3 is caused to project from the forward opening 81 of the catheter 8. Thus, the foreign body capturing part 3, which has been in its elongated state in the catheter 8, automatically deforms (or assumes its natural state) by its own elasticity, as shown in FIG. 6.

The catheter 8 is slightly moved toward the base end so that the forward end of the catheter 8 retreats to a point which is behind the embolus 200. In addition, the wire 1 to remove an intravascular foreign body is also slightly pulled back. This action causes the foreign body capturing part 3 (or the foreign body capturing space 35) to twine around the embolus 200 to capture it, as shown in FIG. 7. In other words, the embolus 200 enters the foreign body capturing space 35 from its right side, as shown in FIG. 7.

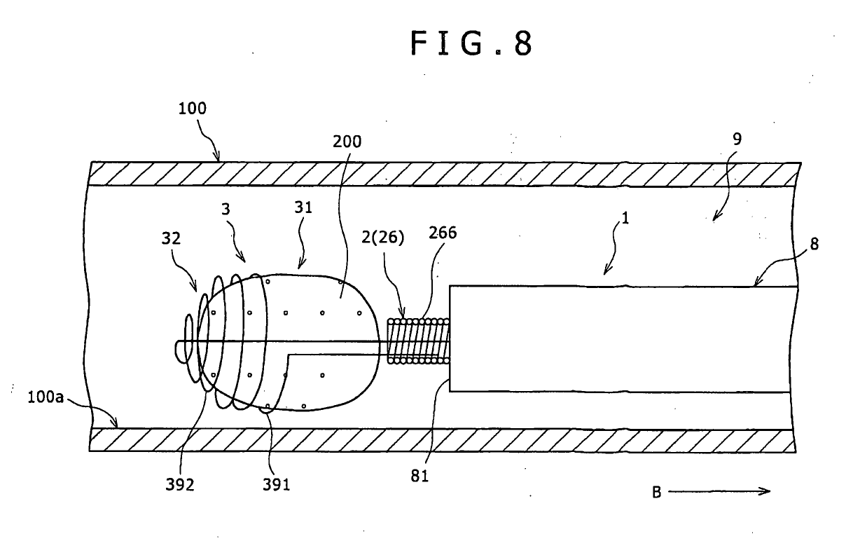

As soon as the embolus 200 is caught in the foreign body capturing space 35, the operating wire 43 is drawn toward the base end of the operating wire 43 (the direction of arrow B in FIG. 8). This operation deforms the second capturing part 32 such that the average distance between adjacent turns of the filament 391 decreases. In other words, the foreign body capturing part 3 assumes its contracted state. The second capturing part 32 is deformed to such an extent that the average distance between adjacent turns of the filament 392 in the second capturing part 32 becomes smaller than the average distance between adjacent turns of the filament 391 in the first capturing part 31.

This operation causes the foreign body capturing space 35 to surely capture the embolus 200. In other words, the embolus 200 is caught in the gap between the filaments 391 and 392 of the foreign body capturing part 3. Therefore, the capturing part 3 can surely capture (hold) the embolus 200.

The second capturing part 32, which has been deformed, functions as the forward wall of the foreign body capturing part 3. This prevents the captured embolus 200 from moving toward the forward end, so that the capturing part surely captures (and holds) the embolus 200.

The wire 1 to remove an intravascular foreign body and the catheter 8 are removed all together, with the above-mentioned holding state maintained. Thus, the embolus 200 is surely captured in the guiding catheter or sheath introducer (not shown).

### Second Embodiment

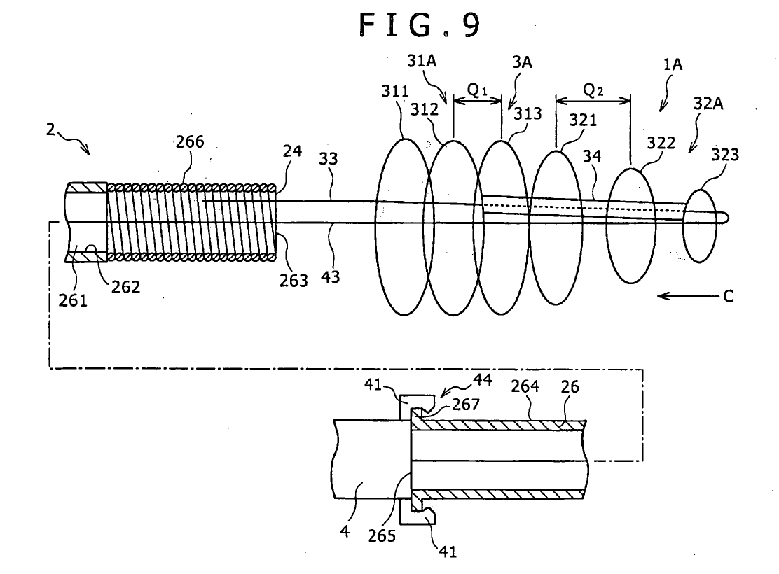

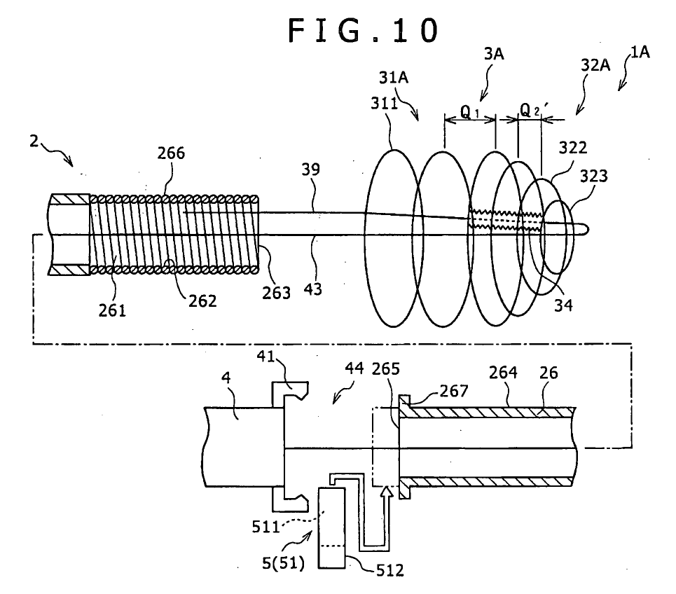

FIG. 9 is a partial longitudinal sectional view showing the wire to remove an intravascular foreign body (in its natural state) pertaining to the second embodiment of the present invention. FIG. 10 is a partial longitudinal sectional view showing the wire to remove an intravascular foreign body (in its contracted state) shown in FIG. 9.

These figures will be referenced in the following description about the wire to remove an intravascular foreign body pertaining to the second embodiment of the present invention. Stress is placed on differences between the first embodiment and the second embodiment, and description of those items common to the two embodiments is omitted.

The second embodiment is identical with the first embodiment except for the construction of the foreign body capturing part. As shown in FIG. 9, the foreign body capturing part 3A of the wire 1A to remove an intravascular foreign body includes a first capturing part 31A (which is at the forward end of the wire body 2) and a second capturing part 32A (which is at the forward end of the first capturing part 31A).

The first capturing part 31A has a first group of annular loop wires (i.e., a group of individual endless loops, such as annular loops, formed by respective wires) 311 to 313 which are arranged at certain intervals in the lengthwise direction of the wire body 2 in the natural state. The loop wires 311 to 313 of the first group are connected to each other by a connecting wire 33.

The second capturing part 32A has a second group of loop wires 321 to 323 which are arranged at certain intervals in the lengthwise direction of the wire body 2 in the natural state. The loop wires 321 to 323 gradually decrease in diameter in going toward the forward end (i.e., going toward to the right in FIG. 9). The loop wires 321 to 323 of the second group are connected to each other by a retractable or stretch connecting pipe 34 formed of an elastic material. An operating wire 43 passes through the pipe 34 and is integral with the connecting wire 33. The connecting wire 33 connects the inner surface of the coil 266 (wire body 2).

The wire 1A to remove an intravascular foreign body works as follows. When a foreign body enters the rearwardly open space formed by the first and second groups of loop wires, the operating wire 43 is operated, causing the pipe 34 to be contracted and, as a result, the second group of loop wires (321 to 323) is compressed, with the distance between adjacent loop wires decreased. In other words, the second capturing part 32A assumes the contracted state. In this way the foreign body capturing part 3 prevents the embolus 200 (which might be comparatively soft) from slipping off through the gap between adjacent loop wires. This ensures the capturing of the embolus 200.

The foreign body capturing part 3A is not specifically restricted in the distance between adjacent loop wires, so long as it assumes a shape suitable to capture the foreign body as desired. The average distance (indicated by Q2 in FIG. 9) between adjacent loop wires 321 to 323 (in the second group) of the second capturing part 32A in its natural state should preferably be larger than the average distance (indicated by Q1 in FIG. 9) between adjacent loop wires 311 to 313 (in the first group) of the first capturing part 31A.

In this way the foreign body capturing part 3 changes from its natural state into its contracted state. In its contracted state, the foreign body capturing part 3 surely captures (removes) the embolus 200.

If the embolus 200 in the cerebral blood vessel is to be captured, the average distance Q2 in the axial direction of the wire body 2 between adjacent loop wires 321 to 323 (of the second group) in the second capturing part 32A in its natural state should preferably be 1 to 20 mm, more preferably 3 to 10 mm.

Also, the average distance Q1 between adjacent loop wires 311 to 313 (of the first group) in the first capturing part 31A in its natural state should preferably be 0.1 to 15 mm, more preferably 1 to 5 mm.

If the embolus 200 is to be captured, the operating wire 43 is operated so that the foreign body capturing part 3A assumes the contracted state. In this case, the second capturing part 32A is deformed to such an extent that the average distance (indicated by Q2 in FIG. 10) between adjacent loop wires 321 to 323 (of the second group) in the second capturing part 32A becomes smaller than the average distance Q1. In this way the foreign body capturing part 3 (or the foreign body capturing space 35) can surely capture the embolus 200 which might be comparatively soft.

If the embolus 200 in the cerebral blood vessel is to be captured, the average distance Q2′ in the second capturing part 32A in its contracted state is not restricted but preferably be about 0.1 to 10 mm, more preferably about 0.1 to 3 mm.

Incidentally, the number of loop wires of the first group is not limited to three; it may be two or four or more.

The number of loop wires of the second group is not limited to three either; it may be two or four or more.

The foregoing is about a wire to remove an intravascular foreign body and the medical instrument according to the present invention. It is not intended to restrict the scope of the present invention. The constituents of the wire and medical instrument may be modified or replaced by (or reinforced with) those which function in the same way.

In the foregoing description, it is assumed that the first capturing part and the second capturing part are formed integrally from the same material. The present invention is not limited to such a construction. The first capturing part and the second capturing part may be formed from the different respective materials.

The second capturing part may be modified such that the loop diameter gradually increases in going toward the forward end instead of gradually decreasing, or modified such that the loop diameter at the center is larger than that at both ends.

The swellable coating may be applied not only to the capturing part 3 but also to the wire body.

The operation of the operating wire is not limited to manipulation by the operating member. It could be accomplished by driving a rack and a pinion attached to the operating wire and the wire body, respectively. This arrangement facilitates the operation of the operating wire.

The first and second capturing parts may exhibit rigidity which gradually changes if they are formed from different respective materials or if they are formed from the same material but they differ in filament diameter. This construction permits the second capturing part to be pulled toward the operator's hand and deformed as desired, with the first capturing part retaining its shape, when the operating member is operated.

The operating member. It could be accomplished by driving a rack and a pinion attached to the operating wire and the wire body, respectively. This arrangement facilitates the operation of the operating wire.

The first and second capturing parts may exhibit rigidity which gradually changes if they are formed from different respective materials or if they are formed from the same material but they differ in filament diameter. This construction permits the second capturing part to be pulled toward the operator's hand and deformed as desired, with the first capturing part retaining its shape, when the operating member is operated.

While preferred embodiments of the present invention have been described using specific terms, such description is for illustrative purposes only, and it is to be understood that changes and variations may be made without departing from the spirit or scope of the following claims.

What is claimed is:

1. A wire to remove an intravascular foreign body comprising:
a flexible long wire body having a forward end;
a foreign body capturing portion for capturing a foreign body in a vessel; and
an operating wire to deform said foreign body capturing portion;
said foreign body capturing portion including a first capturing part and a second capturing part,
said first capturing part being formed from a filament which assumes a helical shape in its natural state and being arranged on the forward end of said wire body,
said second capturing part being formed from a filament which assumes a helical shape in its natural state and being arranged on the forward end of said first capturing part,
said second capturing part being deformable such that the distance between its adjacent filament turns decreases upon operation of said operating wire.

2. The wire to remove an intravascular foreign body as defined in claim 1, wherein the average distance between adjacent filament turns of the second capturing part is larger than the average distance between adjacent filament turns of the first capturing part in their natural state.

3. The wire to remove an intravascular foreign body as defined in claim 1, wherein the second capturing part is deformable to such an extent that the average distance between adjacent filament turns of the second capturing part becomes smaller than the average distance between adjacent filament turns of the first capturing part.

4. The wire to remove an intravascular foreign body as defined in claim 1, wherein the first and second capturing parts together form therein a rearwardly open space for receiving a foreign body.

5. A wire to remove an intravascular foreign body comprising:
a flexible long wire body having a forward end;
a foreign body capturing part portion for capturing a foreign body in a vessel; and
an operating wire to pull said foreign body capturing part;
said foreign body capturing portion including a first capturing part placed at the forward end of the wire body and a second capturing part placed at the forward end of the first capturing part,
said first capturing part in its natural state including a first group of loop wires arranged at certain intervals in a lengthwise direction of the wire body,
said second capturing part in its natural state including a second group of loop wires arranged at certain intervals in the lengthwise direction of the wire body,
said second capturing part being deformable such that the distance between the adjacent loop wires of the second group decreases upon operation of said operating wire.

6. The wire to remove an intravascular foreign body as defined in claim 5, wherein the average distance between adjacent loop wires of the second group of the second capturing part is larger than the average distance between adjacent loop wires of the first group of the first capturing part.

7. The wire to remove an intravascular foreign body as defined in claim 5, wherein the second capturing part is deformable to such an extent that the average distance between adjacent loop wires of the second group becomes smaller than the average distance between adjacent loop wires of the first group.

8. The wire to remove an intravascular foreign body as defined claim 5, wherein the second capturing part gradually decreases in loop diameter in going away from the forward end of the wire body.

9. The wire to remove an intravascular foreign body as defined in claim 5, wherein the first and second capturing parts together form a rearwardly open internal space for receiving a foreign body.

10. A medical instrument comprising the wire to remove an intravascular foreign body as defined in claim 1 or claim 5, in combination with a catheter having a lumen for receiving therein the wire to remove an intravascular foreign body.

11. A wire to remove an intravascular foreign body comprising:
a flexible long wire body having a forward end;
a foreign body capturing portion extending from the forward end for capturing a foreign body; and
an operating wire to deform the foreign body capturing portion;
the foreign body capturing portion including a plurality of loops spaced apart in a lengthwise direction of the wire body and defining a rearwardly open internal space for receiving a foreign body as the foreign body capturing portion is moved rearwardly relative to the foreign body;
wherein the operating wire is operable to draw the loops toward one another to resist escape of the captured foreign body from the space.

12. The wire to remove an intravascular foreign body as defined in claim 11 wherein the loops comprise respective turns of a helical filament structure.

13. The wire to remove an intravascular foreign body as defined in claim 11 wherein the loops comprise respective endless loop wires.

14. A method for removing an intravascular foreign body from a patient's vessel, comprising the steps of:
A) introducing a catheter into the vessel;
B) introducing through the catheter a wire body having at its forward end a foreign body capturing portion comprising a plurality of loops spaced apart in a lengthwise direction of the wire body and defining a rearwardly open space;
C) positioning the foreign body capturing portion forwardly of the foreign body;
D) moving the foreign body capturing portion rearwardly to cause the foreign body to enter and be captured in the space; and
E) manipulating an operating wire operably connected to the loops to draw at least some adjacent loops toward one another to rest escape of the foreign body from the space.

15. The method according to claim 14 wherein step B comprises introducing a wire body whose foreign body capturing portion comprises loops formed by respective turns of a helical filament.

16. The method according to claim 14 wherein step B comprises introducing a wire body whose foreign body capturing portion comprises respective endless loop wires.
